(19) United States
(12) Patent Application Publication
(10) Pub. No.: US 2002/0012941 A1
(43) Pub. Date: Jan. 31, 2002

(54) Method for screening for unknown organisms

(75) Inventor: Lewis S. Gruber, Chicago, IL (US)

Correspondence Address: MARSHALL, O'TOOLE, GERSTEIN, MURRAY & BORUN, 6300 SEARS TOWER, 233 SOUTH WACKER DRIVE, CHICAGO, IL, 60606-6402, US

(73) Assignee: Hyseq, Inc.

(21) Appl. No.: 09/954,744

(22) Filed: Sep. 18, 2001

Related U.S. Application Data
(63) Continuation of application No. 09/531,863, filed on Mar. 21, 2000, now Pat. No. 6,291,186.

(51) Int. Cl. C12Q 1/68; G06F 19/00; G01N 33/48; G01N 33/50

(52) U.S. Cl. 435/6; 702/20

(57) ABSTRACT

A method for identifying a unknown organism by comparison of the nucleotide sequence of nucleic acid in a sample with known nucleotide sequences and a determination that an unknown nucleotide sequence is present in the sample.

### FIELD OF THE INVENTION

The present invention relates in general to methods for screening for a nucleic acid of an organism for which a nucleotide sequence is not known, and in particular to methods employing nucleotide sequencing for identification of organisms.

### BACKGROUND

Nucleotide sequencing provides sequence information with various degrees of redundancy. The information obtained from nucleotide sequencing may be used as a source of primary sequence information about the genomes of organisms, and once the nucleotide sequence is known, may be used as a basis for obtaining expression of sequenced genes and of diagnosis of organisms containing the sequenced genes. However, there are prospective advantages for other uses for nucleotide sequencing which do not require knowledge of the existence of the organism to be sequenced.

### SUMMARY OF THE INVENTION

The present invention provides a method for screening a sample containing a nucleic acid for the presence of an organism for which a nucleotide sequence is not known including: sequencing all nucleic acid in a sample; comparing the nucleotide sequence obtained in sequencing step to nucleotide sequences from known organisms; identifying a continuous run of nucleotide sequence as not corresponding to a known nucleotide sequence; and confirming the continuous run of nucleotide sequence as a nucleotide sequence of an organism for which the nucleotide sequence was not otherwise known.

A method according to the present invention may include a sequencing step including the step of sequencing the nucleic acid by hybridization with probes of known sequence.

Preferably a method according to the present invention includes a confirming step comprising the step of constructing an oligonucleotide probe having a continuous sequence of nucleotides or the complement thereto as found in the unknown sequence but not in known'sequences; exposing, under stringent hybridization conditions, the labeled oligonucleotide probe to a sample suspected of containing the oligonucleotide sequence; and identifying the presence of a previously hybridization complex between the labeled oligonucleotide probe and nucleic acid in the sample. Stringent hybridization conditions are those understood in the art to result in hybridization of probes with perfectly matched, but not mismatched sequences of nucleotides.

A method according to the present invention may further comprise a second comparing step wherein a second continuous run of nucleotide sequence is compared with known nucleotide sequences.

A confirming step according to the present invention may comprise the step of: exposing the sample under stringent hybridization conditions to an oligonucleotide probe complementary to a portion of the unknown nucleotide sequence but not to a known nucleotide sequence; and separating a fraction containing a nucleic acid hybridizing to the labeled oligonucleotide from other fractions of the sample. A method according to the present invention may further include the step of microscopically examining the fraction containing the labeled oligonucleotide probe, sequencing nucleic acid in the fraction containing the labeled oligonucleotide probe, and/or a second exposing step wherein the labeled oligonucleotide probe is exposed under stringent hybridization conditions to a second sample.

A method according to the present invention may include a second exposing step comprising the step of obtaining a sample from a second individual or a second sample from the same individual.

### DETAILED DESCRIPTION

Nucleic acids and methods for isolating and cloning such nucleotide sequencing are well known to those of skill in the art. See e.g., Ausubel et al., Current Protocols in Molecular Biology, Vol. 1-2, John Wiley & Sons Publs. (1989); and Sambrook et al., Molecular Cloning A Laboratory Manual, 2nd Ed., Vols. 1-3, Cold Spring Harbor Press (1989), both of which are incorporated by reference herein.

Sequencing by hybridization (“SBH”) is a well developed technology that may be practiced by a number of methods known to those skilled in the art. Specifically, techniques related to sequencing by hybridization of the following documents is incorporated by reference herein: Drmanac et al., U.S. Pat. No. 5,202,231—Issued Apr. 13, 1993; Drmanac et al., Genomics, 4, 114-128 (1989); Drmanac et al., Proceedings of the First Int'l. Conf. Electrophoresis Supercomputing Human Genome Cantor, DR & Lim HA eds, World Scientific Pub. Co., Singpore, 47-59 (1991); Drmanac et al., Science, 260, 1649-1652 (1993); Lehrach et al., Genome Analysis:Genetic and Physical Mapping, 1, 39-81 (1990), Cold Spring Harbor Laboratory Press; Drmanac et al., Nucl. Acids Res., 4691 (1986); Stevanovic et al., Gene, 79, 139 (1989); Panuesku et al., Mol. Biol. Evol., 1, 607 (1990); Drmanac et al., DNA and Cell Biol., 9, 527 (1990); Nizetic et al., Nucl. Acids Res., 19, 182 (1991); Drmanac et al., J. Biomol. Struct. Dyn., 5, 1085 (1991); Hoheisel et al., Mol. Gen., 4, 125-132 (1991); Strezoska et al., Proc. Nat'l. Acad. Sci. (USA), 88, 10089 (1991); Drmanac et al., Nucl. Acids Res., 19, 5839 (1991); and Drmanac et al., Int. J. Genome Res., 1, 59-79 (1992).

SBH technology may be applied to obtain nucleotide sequence information for all or part of the genomes of known organisms. In this process, a number of oligonucleotide probes of a given length, which may be a 7-mer, are separately exposed under hybridization conditions with a sample to be sequenced. Less than the total number of possible probes of a given length may be employed using various techniques, and exposure under hybridization conditions of probes of more than one length may be employed to improve the results. SBH may be complimented by gel sequencing to obtain all of an unknown sample sequence.

According to the present invention, SBH may be applied to a sample of nucleic acid to determine whether it contains nucleic acid from at one organism for which a nucleotide sequence is unknown. Preferably, the sample may contain more than one genome. The nucleotide sequence obtained for a nucleic acid in the sample may be compared with nucleotide sequences for genomes of known organisms which may be eliminated from consideration. Continuous nucleotides sequence obtain from a sample, which sequence does not correspond to any known nucleotide sequence for a known organism, identifies the presence of a previously unknown organism.

The nucleotide sequence for the previously unknown organism that is obtained by SBH may then be used to make labeled oligonucleotide probes to diagnose the presence or absence of the organism and as an aid in identifying and isolating the previously unknown organism. Techniques such as filtration, centrifugation and chromatography may be applied to separate the organism from otherwise known organisms. Labeled oligonucleotide probes may be used as markers to identify the presence of the previously unknown organism in separatory fractions to obtain purified samples of organisms. Such purified samples of the organism may be sequenced in order to verify the original determination of the presence of a previously unknown organism and to verify the obtaining of a nucleotide sequence for the organism to whatever degree of completeness is desired.

Identification of previously unknown organisms by SBH may be employed in a diagnostic setting for determining organisms responsible for causing disease. Similarly, the method according to the present invention may be applied to identify new organisms in, for example, soil, air and water samples. Such a determination may be used to screen for organisms having a desirable or undesirable effect observed from the soil, air or water sample (such as degradation of pollutants or nitrification). Similarly, organisms having an adverse or beneficial when found effect in food may be detected by using the method of the present invention. For example, where a phenotype is desired, a microorganism which has desirable properties may be identified by SBH even out of a mixture of unknown organisms by correlating presence of hybridization with a labeled probe constructed on the basis from SBH with the presence in a sample of the desired phenotype.

### EXAMPLE

A blood sample from a subject exhibiting disease symptoms screened according to the present invention. Fractions of the blood sample suspected of containing a microorganism which may be responsible for the disease symptoms are preparatively treated to obtain a cDNA library useful for screening. Such preparation may include cloning of the DNA in vectors and amplification of the cloned nucleic acid by PCR.

After application of SBH procedures, sequence information is obtained. The sequence information is in the form of stretches of nucleotide sequence representing the overlapping runs of nucleotide sequence (a run being a continuous sequence formed by overlapping more than one probe sequence) of oligonucleotide probes which hybridize to cloned DNA from the sample. Nucleotide sequences known, e.g., from GENBANK (BBN Laboratories, Inc. 10 Moulton Street, Cambridge, Mass.) or another source of nucleotide sequence information are excluded while the remaining sequences are further examined as follows.

In some instances, sequence from more than two clones may partially overlap, indicating the presence of a branch point. Such a branch point may indicate two similar stretches of nucleotide sequence in an organism in the sample, or may indicate a common portion of a sequence in two or more organisms in the sample. The sequences through each branch of the branch point are compared to known sequences. If the nucleotide sequence of a branch sequence does not correspond to a known nucleotide sequence for an organism, the determination of the nucleotide sequence of the branch is taken as an identification of an unknown organism.

Discontinuous runs of overlapping sequence which do not correspond to a nucleotide sequence from an organism for which a nucleotide sequence is known, may indicate a fragmentary sequence is present or may indicate that more than one organism is present. Such discontinuous runs of sequence are compared with nucleotide sequences from known organisms, and, to the extent that the sequence from the sample does not correspond to a known sequence, presence of at least one organism is identified.

The presence of an unknown organism is verified by synthesizing an oligonucleotide probe corresponding to a unique portion of a continuous run of nucleotide sequence identified as coming from an unknown organism or to the complement of the sequence. Such a probe is applied to the sample to confirm the presence of the determined sequence in the sample. Such a probe is applied to: another sample from the same individual from which the first sample was derived; to a sample from a second individual who has diagnostic disease symptoms similar to those of the first individual; and to a sample from a third individual who does not have diagnostic symptoms similar to those of the first individual. The presence of the nucleotide sequence but in samples from the same and the second individual but not the third individual identifies the sequence as being from a previously unknown organism.

Oligonucleotide probes are made and used to identify a fraction of a sample from an individual identified as containing the nucleic acid above. The contents of the fraction hybridizing to a labeled probe having the same or the complement of a nucleotide sequence of a previously unknown organism are examined using microscopic techniques to visually detect a previously unknown organism. Separatory techniques are applied to fractions containing the previously unknown organism to track the presence of the previously unknown organism through fractions obtain from purification procedures known to those skilled in the art, which procedures separate the previously unknown organism from known organisms in the sample.

Once separated from other organisms in the sample, sequencing by hybridization is applied to obtain a complete nucleotide sequence for the nucleic acid of the previous unknown organism.

The present invention has been described in terms of a particular embodiment. However, it is contemplated that modifications and improvements will occur to those skilled in the art upon consideration of the present specification and claims. For example, although a preferred method using SBH has been exemplified herein, gel sequencing or other nucleotide sequencing techniques may be employed solely or in combination with each other or SBH. Accordingly, it is intended that all variations and modifications of the present invention be included within the scope of the claims.

1. A method for screening a sample containing a nucleic acid for the presence of an organism for which a nucleotide sequence is not known comprising the steps of:
sequencing all nucleic acid in a sample;
comparing the-nucleotide sequence obtained in said sequencing step to nucleotide sequences from known organisms;
identifying a continuous run of nucleotide sequence as not corresponding to a known nucleotide sequence; and
confirming the continuous run of nucleotide sequence as a nucleotide sequence of an organism for which the nucleotide sequence was not otherwise known.

2. The method as recited in claim 1 wherein said sequencing step comprises the step of sequencing the nucleic acid by hybridization with probes of known sequence.

3. The method as recited in claim 1 wherein said confirming step comprises the step of constructing an oligonucleotide probe having a continuous sequence of nucleotides or the complement thereto as found in the unknown sequence but not in known sequences;
exposing, under stringent hybridization conditions, the labeled oligonucleotide probe to a sample suspected of containing the oligonucleotide sequence; and
identifying the presence of a hybridization complex between the labeled oligonucleotide probe and the previously unknown nucleic acid in the sample.

4. The method as recited in claim 1 further comprising a second comparing step wherein a second continuous run of nucleotide sequence is compared with known nucleotide sequences.

5. The method as recited in claim 1 wherein said confirming step comprises the steps of:
exposing the sample under stringent hybridization conditions to an oligonucleotide probe complementary to a portion of said unknown nucleotide sequence but not to a known nucleotide sequence; and
separating a fraction containing a nucleic acid hybridizing to the labeled oligonucleotide from other fractions of the sample.

6. The method as recited in claim 5 further comprising the step of microscopically examining the fraction containing the labeled oligonucleotide probe.

7. The method as recited in claim 5 further comprising the step of sequencing nucleic acid in the fraction containing the labeled oligonucleotide probe.

8. The method as recited in claim 5 further comprising a second exposing step wherein the labeled oligonucleotide probe is exposed under stringent hybridization conditions to a second sample.

9. The method as recited in claim 8 wherein said second exposing step comprises the step of obtaining a sample from a second individual.

10. The method as recited in claim 8 wherein said second exposing step comprises the step of obtaining a second sample from the same individual.
